(19) United States
(12) Patent Application Publication
(10) Pub. No.: US 2005/0131027 A1
(43) Pub. Date: Jun. 16, 2005

(54) Fine particle size pioglitazone

(76) Inventors: Guy Samburski, Hofit (IL); Ben-Zion Dolitzky, Petach Tiqva (IL)

Correspondence Address: KENYON & KENYON, ONE BROADWAY, NEW YORK, NY, 10004, US

(21) Appl. No.: 10/508,741

(22) PCT Filed: Mar. 21, 2003
(86) PCT No.: PCT/US03/08945

Related U.S. Application Data
(60) Provisional application No. 60/366,352, filed on Mar. 21, 2002.

(51) Int. Cl. A61K 31/4439; C07D 417/02

(52) U.S. Cl. 514/340; 546/269.7

(57) ABSTRACT

The present invention provides pioglitazone of defined particle size distribution. The Pioglitazone of defined particle size can be formulated into a wide variety of dosage forms.

### CROSS-REFERENCE TO RELATED APPLICATION

This application claims the benefit under 35 S.C. 119(e) of provisional application Ser. No. 60/366,352, filed Mar. 21, 2002, which is incorporated herein by reference.

### FIELD OF THE INVENTION

The present invention relates to PIOGLITAZONE of defined particle size and oral dosage forms containing pioglitazone of defined particle size.

### BACKGROUND OF THE INVENTION

PIOGLITAZONE HYDROCHLORIDE (hereafter pioglitazone) is an oral antihyperglycemic agent that acts primarily by decreasing insulin resistance. Pharmacological studies indicate that pioglitazone improves sensitivity to insulin in muscle and adipose tissue and inhibits hepatic gluconeogenesis. Pioglitazone improves glucose resistance while reducing circulating insulin levels, and is useful in the treatment of diabetes, particularly type II diabetes. Type II diabetes is known as a disease characterized by insulin resistance.

Pioglitazone is currently marketed as ACTOS®. Pioglitazone hydrochloride has the chemical name [(±)5-[[4-[2-(5-ethyl-2-pyridinyl)ethoxy]phenyl]methyl]-2,4-] thiazolidinedione monohydrochloride. (CAS Registry No. 111025-46-8). The chemical structure of pioglitazone is shown as Formula I. 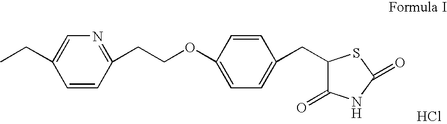
U.S. Pat. No. 5,952,509, incorporated herein by reference, discloses methods for the synthesis of pioglitazone.

Particles size can affect the solubility properties of a compound, like Pioglitazone. Particle size reduction may be tried in order to increase a compound's solubility. Particle size reduction increases the surface area of the solid phase that is in contact with the liquid medium. However, particle size reduction cannot alter the solubility of the compound in a solvent, which is a thermodynamic quantity.

There are instances where the rate of dissolution of a poorly soluble drug is the rate limiting factor in its rate of absorption by the body. It is recognized that such drugs may be more readily bioavailable if administered in a finely divided state.

Particle size also can affect how freely crystals or a powdered form of a drug will flow past each other which has consequences in the production process of pharmaceutical products containing the drug.

In view of the foregoing, there is a need in the medical arts for Pioglitazone with a small particle size and improved bioavailability.

### SUMMARY OF THE INVENTION

In one aspect, the present invention relates to pioglitazone of defined particle size including a plurality of pioglitazone particles wherein the mean particle size (d₀₀₅) is about 2 μm to about 7 μm and 10 volume percent or less of the plurality of particles have a particle diameter equal to or greater than about 10 μm.

In another aspect, the present invention relates to pioglitazone of defined particle size including a plurality of pioglitazone particles obtained by comminution using a fluid energy mill, wherein the mean particle size (d₀₀₅) is about 2 μm to about 7 μm and 10 volume percent or less of the plurality of particles have a particle diameter equal to or greater than about 10 μm.

In another aspect, the present invention relates to a pharmaceutical composition including pioglitazone of defined particle size, wherein the mean particle size (d_{.05}) is about 2 μm to about 7 μm and 10 volume percent or less of the plurality of particles have a particle diameter equal to or greater than about 10 μm, and at least one pharmaceutically acceptable excipient, especially a pharmaceutically acceptable excipient selected from the group consisting of microcrystalline cellulose, microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylate, potassium chloride, powdered cellulose, sodium chloride, sorbitol, talc, acacia, alginic acid, carbomer, carboxymethylcellulose sodium, dextrin, ethyl cellulose, gelatin, guar gum, hydrogenated vegetable oil, hydroxyethyl cellulose, hydroxypropyl cellulose, hydroxypropyl methylcellulose, liquid glucose, magnesium aluminum silicate, maltodextrin, methylcellulose, polymethacrylates, povidone, pregelatinized starch, sodium alginate, starch, alginic acid, carboxymethyl cellulose calcium, colloidal silicon dioxide, croscarmellose sodium, crospovidone, guar gum, magnesium aluminum silicate, methyl cellulose, polacrilin potassium, powdered cellulose, pregelatinized starch, sodium alginate and sodium starch glycolate.

In yet another aspect, the present invention relates to a pharmaceutical composition including pioglitazone of defined particle size, wherein the mean particle size (d_{.05}) is about 2 μm to about 7 μm and 10 volume percent or less of the plurality of particles have a particle diameter equal to or greater than about 10 μm, and at least one pharmaceutically acceptable excipient, wherein the pharmaceutical composition is in the form of an oral solid dosage form, especially a tablet or capsule.

In a further aspect, the present invention relates to an oral liquid dosage form including pioglitazone of defined particle size, wherein the mean particle size (d_{.05}) is about 2 μm to about 7 μm and 10 volume percent or less of the plurality of particles have a particle diameter equal to or greater than about 10 μm, and a pharmaceutically acceptable liquid carrier, especially a pharmaceutically acceptable liquid carrier selected from the group consisting of water, vegetable oil, alcohol, polyethylene glycol, propylene glycol and glycerin.

In still another aspect, the present invention relates to a method of treating a disease selected from hyperglycemia, insulin resistance, and diabetes including the step of administering to a mammal, especially a human, in need of treatment for one of the diseases a solid oral dosage form, especially a tablet or capsule, including pioglitazone of defined particle size, wherein the mean particle size (d_{.05}) is about 2 μm to about 7 μm and 10 volume percent or less of the plurality of particles have a particle diameter equal to or greater than about 10 μm, especially wherein the particles are obtained by use of a fluid energy mill, and at least one pharmaceutically acceptable excipient, especially a pharmaceutically acceptable excipient selected from the group consisting of microcrystalline cellulose, microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylate, potassium chloride, powdered cellulose, sodium chloride, sorbitol, talc, acacia, alginic acid, carbomer, carboxymethylcellulose sodium, dextrin, ethyl cellulose, gelatin, guar gum, hydrogenated vegetable oil, hydroxyethyl cellulose, hydroxypropyl cellulose, hydroxypropyl methylcellulose, liquid glucose, magnesium aluminum silicate, maltodextrin, methylcellulose, polymethacrylates, povidone, pregelatinized starch, sodium alginate, starch, alginic acid, carboxymethyl cellulose calcium, colloidal silicon dioxide, croscarmellose sodium, crospovidone, guar gum, magnesium aluminum silicate, methyl cellulose, polacrilin potassium, powdered cellulose, pregelatinized starch, sodium alginate and sodium starch glycolate.

In still a further aspect, the present invention relates to a method of treating a disease selected from hyperglycemia, insulin resistance, and diabetes including the step of administering to a mammal, especially a human, in need of treatment for one of the diseases, an oral liquid dosage form including pioglitazone of defined particle size, wherein the mean particle size (d_{.05}) is about 2 μm to about 7 μm and 10 volume percent or less of the plurality of particles have a particle diameter equal to or greater than about 10 μm, especially wherein the particles are obtained by use of a fluid energy mill, and a pharmaceutically acceptable liquid carrier, especially a pharmaceutically acceptable liquid carrier selected from the group consisting of water, vegetable oil, alcohol, polyethylene glycol, propylene glycol and glycerin.

### DETAILED DESCRIPTION OF THE INVENTION

The present invention provides Pioglitazone of defined particle size distribution The pioglitazone of defined particle size of this invention comprises a plurality of pioglitazone particles. Individual particles of the plurality will vary in characteristics and the characteristics of no individual or small proportion of the particles will materially affect the properties of the bulk material. Rather, the characteristics of the pioglitazone are determined from a statistically significant sampling and measurement of bulk, or mean, properties of the sample. Statistically significant measurements include those with a statistical sampling error of about 2% or less.

The Pioglitazone of defined particle size is useful for preparing pharmaceutical compositions and compressed solid dosage forms, encapsulated free flowing and compressed dosage forms, enteral solutions, suspensions and elixers.

As used herein, pharmaceutical composition means a composition (medicament) for use in treating a mammal that includes pioglitazone of defined particle size and is prepared in a manner that is appropriate for administration to a mammal, preferably a human. A pharmaceutical composition also can and preferably does contain one or more pharmaceutically acceptable excipients, i.e. excipients that are non-toxic (benign) to the mammal intended to be treated when the composition is administered in an amount effective to treat the mammal. The term pharmaceutical composition also includes feedstocks for preparing oral solid or liquid pharmaceutical dosage forms such as tablets, capsules, suspensions and solutions.

As used herein, the terms “median” and “mean” are used interchangeably and, when used in reference to the size of pioglitazone particles, indicate that about 50 volume % of all measurable particles measured have a particle size less than the defined median particle size value, and that about 50 volume % of all measurable particles measured have a particle size greater than the defined median particle size value.

In accordance with the invention, the pioglitazone of defined particle size includes a plurality of Pioglitazone particles characterized by a distribution in which the mean particle diameter of the particles is from about 2 to about 7 μm and 10% or fewer of the particles have a particle diameter equal to or more than about 10 μm.

In this disclosure, the phrase “equal to or less than about” when referring to a particle's diameter encompasses particles that pass through a standard test sieve whose opening size designation is the most proximate to the diameter recited, selected from among sieves whose opening size designations are greater than the recited particle diameter.

The phrase “equal to or more than about” when referring to a particle's diameter encompasses particles that are captured (do not pass through) an ASTM Standard Test Sieve whose opening size designation is the most proximate to the diameter specified, selected from among the Standard Test Sieves whose designations are less than the specified particle diameter.

Pioglitazone of defined particle size can also be produced by precipitation from appropriate solvents. Precipitation and hence particle size can be controlled by customary methods such as cooling, pH adjustment, pouring a concentrated solution of pioglitazone into an anti-solvent and/or by co-precipitation so as to obtain a precipitate with the appropriate average surface area.

Pioglitazone of defined particle size may be produced by known methods of particle size reduction starting with crystals, powder aggregates, and coarse powder of either crystalline or amorphous Pioglitazone. The principal operations of conventional size reduction are milling of a feedstock material and sorting of the milled material by size.

A fluid energy mill, or “micronizer”, is an especially preferred type of mill for its ability to produce particles of small size in a narrow size distribution. As those skilled in the art are aware, micronizers use the kinetic energy of collision between particles suspended in a rapidly moving fluid (typically air) stream to cleave the particles. An air jet mill is a preferred fluid energy mill. The suspended particles are injected under pressure into a recirculating particle stream. Smaller particles are carried aloft inside the mill and swept into a vent connected to a particle size classifier such as a cyclone. The feedstock should first be milled to about 150 to 850 μm which may be done using a conventional ball, roller, or hammer mill.

The most widely practiced method of sorting by particle size involves passing the milled material through a stack of sieves, each with openings of a different size. The sieves are arranged so that the material encounters the sieve having the largest openings first and those particles that pass through the first sieve encounter a second sieve with smaller openings and those that pass through the second sieve may encounter a third sieve, and so forth. Pioglitazone particles can also be separated by particle size using cyclonic or centrifugation techniques.

The size distribution of pioglitazone particles of the present invention is preferably determined by laser diffraction. The size of pioglitazone particles reported herein was determined using a Malvern™ Mastersizer laser diffraction instrument (Malvern Instruments Ltd., Wocestershire, UK). Samples of the pioglitazone were suspended in hexane containing a surfactant, 1% Tween® 80. The suspensions were mixed and then sonicated for 120 seconds to thoroughly disperse the pioglitazone particles. The dispersion was then circulated in the flow cell of the Malvern Mastersizer for two minutes before particle size measurements were taken.

The pharmaceutical compositions of the present invention include pioglitazone of defined particle size and, optionally, one or more pharmaceutically acceptable excipients. Pharmaceutical composition of this invention can be formulated into a variety of oral solid and oral liquid dosage forms for administration to humans and animals. Oral solid dosage forms include tablets, powders, capsules, troches and losenges. Oral liquid dosage forms include suspensions, syrups and elixirs. The most suitable dosage form in any given case will depend on the nature and severity of the condition being treated and other circumstances that will be assessed by the caregiver.

In particular embodiments, the pharmaceutical compositions of the present invention are made into oral solid dosage forms. In this case, the pharmaceutical composition includes one or more pharmaceutically acceptable excipients. Pharmaceutically acceptable excipients are benign, i.e. they are nontoxic to the patient to whom the pharmaceutical composition is administered. Pharmaceutically acceptable excipients are well known in the art and perform various functions. For example they add bulk or act as diluents, improve bulk handling properties, or aid in dissolution or disintergration of the final oral solid dosage form. The skilled artisan knows that a given pharmaceutically acceptable excipient more than one of the foregoing characteristics or properties and classification of excipients according to function is therefore somewhat arbitrary.

The pharmaceutical compositions of the present invention may contain one or more diluents added to make the tablet larger and, hence, easier for the patient and caregiver to handle. Common diluents are microcrystalline cellulose (e.g. Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit), potassium chloride, powdered cellulose, sodium chloride, sorbitol and talc.

Binders also can be included in the pharmaceutical compositions of the present invention to help hold the tablet together after compression. Some typical binders are acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon°, Plasdone®), pregelatinized starch, sodium alginate and starch.

The pharmaceutical compositions to be made into tablets can further include a disintegrant to accelerate disintegration of the tablet in the patient's stomach. Disintegrants include alginic acid, carboxymethyl cellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

A pharmaceutical composition for tableting may further include glidants, lubricants, flavorings, colorants and other commonly used excipients.

In other embodiments, the pharmaceutical compositions of the present invention are filled into capsules (e.g. hard gelatine capsules). Pharmaceutical compositions to be filled into capsules can and preferably do include pharmaceutically acceptable excipients; for example diluents such as lactose, mannitol, calcium carbonate, or magnesium carbonate; or flow aids such as the stearates.

Preferably, solid oral dosage forms of the present invention will be preferably formulated to provide a unit dose of pioglitazone of about 5 to about 50 milligrams per individual dosage form.

In other embodiments of the present invention, the pioglitazone of defined particle size of the present invention is formulated into an oral liquid dosage form, preferably a suspension or dispersion, that includes a pharmaceutically acceptable liquid vehicle (carrier).

Pharmaceutically acceptable carriers suitable for use in the present invention include water, vegetable oil, alcohol, polyethylene glycol, propylene glycol or glycerin, most preferably water.

Oral liquid dosage forms can contain emulsifying or suspending agents to disperse uniformly throughout the composition the active ingredient or other excipient that has low solubility in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Oral liquid dosage forms of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

The oral liquid dosage form (pharmaceutical composition) can also contain sweetening agents, such as sorbitol, saccharin, sodium saccharin, sucrose, aspartame, fructose, mannitol and invert sugar; preservatives and chelating agents such as alcohol, sodium benzoate, butylated hydroxy toluene, butylated hydroxyanisole and ethylenediamine tetraacetic acid; and buffers such as guconic acid, lactic acid, citric acid or acetic acid, sodium guconate, sodium lactate, sodium citrate or sodium acetate.

In still other embodiments, the present invention provides a method of treating diabetes, hypoglycemia, or insulin resistance by administering an oral solid dosage form or an oral liquid dosage form of the present invention. The medical practitioner will know to adjust the number and frequency of administration of dosage forms of the present invention based on clinical findings and guidance from the medical literature.

1. Pioglitazone of defined particle size comprising a plurality of pioglotazone particles wherein the mean particle size (d_{.05}) is about 2 μm to about 7 μm and 10 volume percent or less of the plurality of particles have a particle diameter equal to or greater than about 10 μm.

2. The pioglitazone of defined particle size of claim 1 obtained by comminution using a fluid energy mill.

3. A pharmaceutical composition comprising the pioglitazone of defined particle size of claim 1 and at least one pharmaceutically acceptable excipient.

4. The pharmaceutical composition of claim 3 wherein the pharmaceutically acceptable excipient is selected from the group consisting of microcrystalline cellulose, microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylate, potassium chloride, powdered cellulose, sodium chloride, sorbitol, talc, acacia, alginic acid, carbomer, carboxymethylcellulose sodium, dextrin, ethyl cellulose, gelatin, guar gum, hydrogenated vegetable oil, hydroxyethyl cellulose, hydroxypropyl cellulose, hydroxypropyl methylcellulose, liquid glucose, magnesium aluminum silicate, maltodextrin, methylcellulose, polymethacrylates, povidone, pregelatinized starch, sodium alginate, starch, alginic acid, carboxymethyl cellulose calcium, colloidal silicon dioxide, croscarmellose sodium, crospovidone, guar gum, magnesium aluminum silicate, methyl cellulose, polacrilin potassium, powdered cellulose, pregelatinized starch, sodium alginate and sodium starch glycolate.

5. The pharmaceutical composition of claim 4 in the form of an oral solid dosage form.

6. The pharmaceutical composition of claim 5 wherein the oral solid dosage form is a tablet.

7. The pharmaceutical composition of claim 5 wherein the oral solid dosage form is a capsule.

8. A method of treating a disease selected from diabetes, hyperglycemia, and insulin resistance comprising administering to a mammal suffering from such disease an oral solid dosage form of claim 5.

9. An oral liquid dosage form comprising the pioglitazone of defined particle size of claim 1 and a pharmaceutically acceptable liquid vehicle.

10. The oral liquid dosage form of claim 9 wherein the pharmaceutically acceptable vehicle is selected from the group consisting of water, vegetable oil, alcohol, polyethylene glycol, propylene glycol and glycerin.

11. The oral solid dosage form of claim 10 wherein the pharmaceutically acceptable vehicle is water.

12. The oral liquid dosage form of claim 9 further comprising a pharmaceutically acceptable additive selected from the group consisting of gelatin, egg yolk, casein, cholesterol, acacia, tragacanth, chondrus, pectin, methyl cellulose, carbomer, cetostearyl alcohol, cetyl alcohol, alginic acid bentonite, carbomer, carboxymethylcellulose calcium or sodium, ethylcellulose, gelatin guar gum, hydroxyethyl cellulose, hydroxypropyl cellulose, hydroxypropyl methylcellulose, maltodextrin, polyvinyl alcohol, povidone, propylene carbonate, propylene glycol alginate, sodium alginate, sodium starch glycolate, starch tragacanth, xanthan gum, sorbitol, saccharin, sodium saccharin, sucrose, aspartame, fructose, mannitol, invert sugar; ethyl alcohol, sodium benzoate, butylated hydroxy toluene, butylated hydroxyanisole, ethylenediamine tetraacetic acid, guconic acid, lactic acid, citric acid, acetic acid, sodium guconate, sodium lactate, sodium citrate and sodium acetate.

13. A method of treating a disease selected from diabetes, hyperglycemia, and insulin resistance comprising the step of administering to a mammal suffering from such disease and oral liquid dosage form of claim 9.
